(19) United States
(12) Patent Application Publication
(10) Pub. No.: US 2011/0097368 A1
(43) Pub. Date: Apr. 28, 2011

(54) Treatment Varnish Compositions for Teeth Surfaces

(75) Inventor: Steven D. Jensen, South Jordan, UT (US)

(73) Assignee: CAO GROUP, INC., West Jordan, UT (US)

(21) Appl. No.: 12/911,622

(22) Filed: Oct. 25, 2010

Related U.S. Application Data
(60) Provisional application No. 61/279,611, filed on Oct. 23, 2009.

(51) Int. Cl. A61K 8/02; A61K 8/81; A61Q 11/00

(52) U.S. Cl. 424/401; 424/49; 424/52

(57) ABSTRACT

Embodiments of the present invention include compositions, methods, and systems for providing an effective and efficient treatment varnish for teeth. For example, embodiments of the present invention provide a treatment varnish that comprises one or more polymers and/or copolymers that are soluble in biocompatible solvents, such as ethanol. The polymers and/or copolymers are nontoxic and form a substantially clear film that adheres effectively to teeth during treatment, but also is easily removed from teeth once the treatment is complete.

### CROSS-REFERENCE TO RELATED APPLICATION

This application claims the benefit of U.S. Provisional Application No. 61/279,611, filed Oct. 23, 2009, which is incorporated herein by reference in its entirety.

### TECHNICAL FIELD OF THE INVENTION

The present disclosure generally relates to chemical compositions used for various dental treatments.

### BACKGROUND OF THE INVENTION

Dental professionals, and dental product manufactures, have traditionally used fluoride in an effort to prevent tooth decay. The chemistry behind why fluoride prevents tooth decay is relatively straightforward. Generally, the beneficial function of the fluoride ion occurs during the natural re-mineralization cycles that take place between the teeth and saliva. In particular, the fluoride ion is more electronegative than the hydroxide ion, and therefore will convert calcium hydroxyapatite on the tooth surface to calcium fluoroapatite, which is significantly more resistant to acid. Thus, by forming a layer of calcium fluoroapatite on the surface of the tooth, fluoride treatments can prevent tooth decay.

Although the science behind conventional fluoride treatments is relatively well known, the manner in which the fluoride is actually delivered to the teeth presents a wide array of difficult challenges. For example, one conventional manner to treat teeth with fluoride is to provide toothpaste that contains fluoride. Fluoride toothpaste, however, has several drawbacks that limit the effectiveness of the fluoride treatment. For example, in order for the fluoride in the toothpaste to provide an effective treatment, there needs to be a sufficient amount of contact time between the fluoride toothpaste and the teeth. Many people have inconsistent brushing habits, which leads to ineffective results.

For example, the average individual in the United States may brush their teeth two times a day, or less. Moreover, the average individual in the United States, when they do brush their teeth, may only brush for sixty seconds or less. In contrast, fluoride uptake into enamel is a relatively slow process and takes a longer period of contact than the sixty seconds that is provided when brushing. Although residual fluoride may remain in the saliva after brushing and continue to provide fluoride treatment to the teeth, most individuals rinse their mouth out with water immediately after brushing and essentially rid the saliva of any residual fluoride. In general, a fluoride treatment plan that relies on fluoride toothpaste has many drawbacks that reduce the effectiveness of the fluoride treatment, and therefore, reduce the amount of tooth decay that is prevented.

Dental professionals are well aware that many of their patients do not receive an effective fluoride treatment from daily brushing, especially young patients. Dental professionals combat this deficiency with powerful treatments of fluoride during annual or bi-annual checkups. Fluoride treatments given during a dental checkup attempt to deliver the maximum dose of fluoride to the teeth in a short period of time so that the patient's teeth at least has some tooth decay prevention between dental visits. The typical fluoride delivery method in a dental office may include a disposable dental tray that is filled with a high dosage of fluoride gel, which is then placed onto the patient's teeth for a limited time. Some formulations of the fluoride gel even acidulate the fluoride to increase activity because of the limited time of application in the dental chair.

Although the method of using fluoride gels and fluoride trays provides an effective single dose of fluoride, the method has several drawbacks. For example, fluoride trays may require significant quantities of gel to adequately fill the fluoride tray so that the patient's teeth are covered with the fluoride gel. Fluoride gels are toxic and, due to the large quantities of fluoride gel used in connection with fluoride trays, present a significant toxicity threat to the patient. Thus, dental professions must carefully monitor the patient during treatment to ensure the patient doesn't swallow significant quantities of the fluoride gel in an attempt to prevent accidental poisoning. Additionally, dental professionals must carefully remove the tray and rinse any residual gel from the teeth into a suction device to further avoid the toxicity threat.

An example improvement to fluoride treatments over the fluoride tray is fluoride varnish. Typically, a fluoride varnish includes a coating composition that contains a concentration of fluoride. A dental professional may apply the fluoride varnish to a patient's teeth. The dental professional can then solidify the fluoride varnish by evaporating the solvents with an air syringe until the fluoride varnish becomes a solid hard coat on the patient's teeth. The fluoride varnish coating may release fluoride onto the teeth and into the saliva for about one to two hours before becoming depleted of fluoride.

Fluoride varnishes have many advantages as a fluoride treatment method compared to toothpaste or fluoride trays. For example, fluoride varnishes maximize the time period in which the teeth remain in contact with the fluoride, while at the same time minimizing exposure to large quantities of high dose fluoride gels. A fluoride varnish may also minimize chair time, since after a relatively quick application process the patient may simply leave.

Although fluoride varnishes represent a significant improvement over fluoride toothpaste and fluoride trays, conventional fluoride varnishes have many disadvantages that may reduce the effectiveness of the fluoride treatment. For example, some conventional fluoride varnishes may be made with tree rosins and resins that are partially dissolved by alkane solvents such as hexane or heptane. These rosin coatings are hydrophobic and do not release sufficient fluoride in an effective manner. Moreover, solvents such as hexane or heptanes are not very biocompatible. Other conventional fluoride varnishes contain polymers dissolved in solvent such as ethyl or butyl acetate. Ethyl and butyl acetate are harsh on oral tissue, and are barely tolerable by the patient.

In addition to the harsh and/or anti-biocompatible solvents used in many conventional fluoride varnishes, many conventional fluoride varnishes leave a long lasting hard coat on the teeth that must be broken and picked from the teeth. For example, some conventional fluoride varnishes leave a hard coat on the teeth that lasts for days, long after the two hours of fluoride release has occurred.

Moreover, many conventional fluoride varnishes may have a yellow color and are not aesthetically pleasing to the patient, causing the patient to remove the fluoride varnish as soon as possible and before the fluoride treatment is complete.

Furthermore, the fluoride concentrations of conventional fluoride varnishes are typically in the range of 5% sodium fluoride. Due to the fact that 4.1 grams of sodium fluoride is soluble in 100 grams water the fluoride varnish is often a suspension. To avoid this difficulty, many manufacturers use an organic form of fluoride, however, the organic form of fluoride releases fluoride ions at a much slower rate.

Accordingly, there are a number of disadvantages in the conventional art of providing an effective treatment varnish for teeth.

### SUMMARY OF THE INVENTION

Embodiments of the present invention include compositions, methods, and systems for providing an effective and efficient treatment varnish for teeth. For example, embodiments of the present invention provide a treatment varnish that comprises one or more polymers and/or copolymers that are soluble in biocompatible solvents, such as ethanol. The polymers and/or copolymers are nontoxic and form a substantially clear film that adheres effectively to teeth during treatment, but also is easily removed from teeth once the treatment is complete. Preferred polymers and/or copolymers also form an open matrix when dried (e.g., the solvent is removed) such that water (e.g., saliva) can infiltrate the resulting film and release one or more imbedded active ingredients at a consistent rate over a period of time.

Additional features and advantages of the invention will be set forth in the description which follows, and in part will be obvious from the description, or may be learned by the practice of the invention. The features and advantages of the invention may be realized and obtained by means of the instruments and combinations particularly pointed out in the appended claims. These and other features of the present invention will become more fully apparent from the following description and appended claims, or may be learned by the practice of the invention as set forth hereinafter.

### DETAILED DESCRIPTION OF THE INVENTION

Embodiments of the present invention include compositions, methods, and systems for providing an effective and efficient treatment varnish for teeth. For example, embodiments of the present invention provide a treatment varnish that comprises one or more polymers and/or copolymers that are soluble in biocompatible solvents, such as ethanol. The polymers and/or copolymers are nontoxic and form a substantially clear film that adheres effectively to teeth during treatment, but also is easily removed from teeth once the treatment is complete. Preferred polymers and/or copolymers also form an open matrix when dried (e.g., the solvent is removed) such that water (e.g., saliva) can infiltrate the resulting film and release one or more imbedded active ingredients at a consistent rate over a period of time.

In general, a treatment varnish can be prepared by dissolving a polymer and/or copolymers into a biocompatible solvent. Depending on the type of treatment desired, one or more active ingredients can be added to the mixture (e.g., fluoride, re-mineralizers, periodontal treatments, and/or desensitizers). For example, a combination of various active ingredients can be combined to create a multi-treatment varnish. The mixture can also contain minor amounts of preservatives, flavors, and colorants as desired or needed.

As briefly discussed above, the treatment varnish can comprise various polymers and/or copolymers. In one example embodiment, a treatment varnish for teeth comprises polymers such as poly vinyl butyral and its various copolymers. In particular, example embodiments of the present invention can comprise one or more copolymers of poly vinyl butyral that are soluble in ethanol, but not soluble in water (or at least dissolve at a slow rate in water). Other example polymers can include poly vinyl alcohol, poly vinyl acetate, and similar polymers, which are within the scope of the present invention. Compositions that incorporate combinations of one or more of these polymers are also within the scope of the present invention.

In another example embodiment, a treatment varnish can comprise the copolymers poly(vinyl butyral-co-vinyl alcohol-co-vinyl acetate). These copolymers are especially useful because the copolymers have a large chain and high molecular weight, yet are soluble in ethanol. In particular, treatment varnishes utilizing polymers and/or copolymers with a molecular weight greater than 150,000 form ideal films when dried from ethanol solvent based compositions, as will be discussed further below.

Examples of fluoride sources include both inorganic and organic compounds, which include, but are not limited to, sodium fluoride, potassium fluoride, tetrabutylammonium tetrafluoroborate, and/or sodium monofluorophosphate.

Examples of re-mineralizers include, but are not limited to, calcium phosphate monobasic, calcium phosphate dibasic, calcium ascorbate, calcium phosphate tribasic, sodium fluoride, novamin, potassium fluoride and/or other useful compounds.

Examples of desensitizers include, but are not limited to, potassium nitrate, calcium citrate, calcium sucrose phosphate, glutaraldehyde and/or similar agents.

Examples of periodontal compounds include, but are not limited to, chlorhexidine gluconate, glutaraldehyde, chlorhexidine, sodium chlorate, ubidicarinone, sodium hypochlorite, aloe, bromelain, rhubarb extract and/or other similar useful compounds.

As discussed, one example solvent is ethanol. Ethanol functions as a useful solvent because ethanol is both biocompatible and is not harsh on the oral tissue within the mouth. Other solvents with similar characteristics may also be utilized.

The below list of examples illustrate various example treatment varnish compositions according to the present invention. The percentages represent approximate weight percentages used for illustrative purposes, and therefore are in no way limited to the exact weight percentage shown. The actual weight percentage of any given ingredient in any of the below compositions can vary from one embodiment to the next. Moreover, the examples presented are by no means representative of all the various combinations and formulations that are possible by the practice of the present invention.

### Example 1

### Fluoride Treatment Varnish

| Component | % by Weight |
|---|---|
| Sodium Fluoride | 1% |
| Poly(vinyl butyral-co-vinyl alcohol-co- | 7% |
| vinyl acetate) MW 170,000-250,000 | |
| Peppermint Oil | 0.3% |
| Sucralose | 0.2% |
| Ethanol | 71.5% |
| Water | 20% |

### Example 2

### Fluoride Treatment Varnish in Suspension

| Component | % by Weight |
|---|---|
| Potassium Flouride | 5% |
| Poly(vinyl butyral-co-vinyl alcohol-co- | 7.5% |
| vinyl acetate) MW 170,000-250,000 | |
| Peppermint Oil | 0.3% |
| Sucralose | 0.2% |
| Ethanol | 87% |

### Example 3

### Fluoride Treatment Varnish in Suspension w/ Re-Mineralizer

| Component | % by Weight |
|---|---|
| Sodium Fluoride | 5% |
| Poly(vinyl butyral-co-vinyl alcohol-co- | 6% |
| vinyl acetate) MW 170,000-250,000 | |
| Peppermint Oil | 0.3% |
| Sucralose | 0.2% |
| Ethanol | 78.5% |
| Novamin | 10% |

### Example 4

### Re-Mineralizer Treatment Varnish in Suspension

| Component | % by Weight |
|---|---|
| Poly(vinyl butyral-co-vinyl alcohol-co- | 6% |
| vinyl acetate) MW 170,000-250,000 | |
| Peppermint Oil | 0.3% |
| Sucralose | 0.2% |
| Ethanol | 83.5% |
| Novamin | 10% |

### Example 5

### Fluoride Treatment Varnish w/ Re-Mineralizer in Suspension

| Component | % by Weight |
|---|---|
| Sodium Fluoride | 5% |
| Poly(vinyl butyral-co-vinyl alcohol-co- | 6% |
| vinyl acetate) MW 170,000-250,000 | |
| Peppermint Oil | 0.3% |
| Sucralose | 0.2% |
| Ethanol | 78.5% |
| Calcium Phosphate Monobasic | 10% |

### Example 6

### Fluoride Treatment Varnish w/ Re-Mineralizer in Suspension

| Component | % by Weight |
|---|---|
| Sodium Fluoride | 5% |
| Poly(vinyl butyral-co-vinyl alcohol-co- | 6% |
| vinyl acetate) MW 170,000-250,000 | |
| Peppermint Oil | 0.3% |
| Sucralose | 0.2% |
| Ethanol | 78.5% |
| Water | 10% |

### Example 7

### Fluoride Treatment Varnish w/ Re-Mineralizer in Suspension

| Component | % by Weight |
|---|---|
| Sodium Fluoride | 5% |
| Poly(vinyl butyral-co-vinyl alcohol-co- | 6% |
| vinyl acetate) MW 170,000-250,000 | |
| Peppermint Oil | 0.3% |
| Sucralose | 0.2% |
| Ethanol | 78.5% |
| Calcium Phosphate Tribasic | 10% |

### Example 8

### Desensitizing Treatment Varnish

| Component | % by Weight |
|---|---|
| Potassium Nitrate | 5% |
| Poly(vinyl butyral-co-vinyl alcohol-co- | 7% |
| vinyl acetate) MW 170,000-250,000 | |
| Peppermint Oil | 0.3% |
| Sucralose | 0.2% |
| Ethanol | 67.5% |
| Water | 20% |

### Example 9

### Periodontal and Desensitizing Treatment Varnish

| Component | % by Weight |
|---|---|
| Glutaraldehyde | 5% |
| Poly(vinyl butyral-co-vinyl alcohol-co- | 7% |
| vinyl acetate) MW 170,000-250,000 | |
| Peppermint Oil | 0.3% |
| Sucralose | 0.2% |
| Ethanol | 87.5% |

### Example 10

### Fluoride Treatment Varnish

| Component | % by Weight |
|---|---|
| Tetrabutylammonium Tetrafluoroborate | 5% |
| Poly(vinyl butyral-co-vinyl alcohol-co- | 7% |
| vinyl acetate) MW 170,000-250,000 | |
| Peppermint Oil | 0.3% |
| Sucralose | 0.2% |
| Ethanol | 87.5% |

### Example 11

### Periodontal Treatment Varnish

| Component | % by Weight |
|---|---|
| Chlorhexidine Gluconate | 3% |
| Poly(vinyl butyral-co-vinyl alcohol-co- | 3% |
| vinyl acetate) MW 170,000-250,000 | |
| Peppermint Oil | 0.3% |
| Sucralose | 0.2% |
| Ethanol | 93.5% |

As illustrated in the above examples, the treatment varnish compositions can be prepared in a composition that is ready to apply. Alternatively, the treatment varnish compositions can be prepared in suspension such that it is necessary to mix the suspended treatment varnish composition prior to use.

When the treatment varnish is ready to apply, a dental professional can apply the treatment varnish to the teeth of a patient using small brush or similar item to completely coat the teeth with the treatment varnish. The dental professional can then dry the treatment varnish (e.g., evaporate the solvent out of the composition) by allowing the varnish to simply air dry, or by blowing air on the treatment varnish with an air syringe. Once dry, the treatment varnish forms a treatment film on the teeth that allows the active ingredient(s) to leach out of the treatment varnish composition, and onto the teeth and/or into the saliva.

The above examples exhibit one or more characteristics that are contrary to the characteristics of conventional treatment varnishes. For example, poly vinyl butyral and its copolymers are soluble in ethanol, but not soluble in water. This allows the treatment varnish to utilize a solvent that is not harsh on the tissue of the mouth, yet at the same time still form an effective treatment film on the teeth because the poly vinyl butyral and its copolymers are not soluble in saliva.

Additionally, unlike convention treatment varnishes, treatment varnishes derived from poly(vinyl butyral-co-vinyl alcohol-co-vinyl acetate) form a semi-translucent treatment film when dried from ethanol solutions. The semi-translucent treatment film is aesthetically pleasing in that it is difficult for others to see or notice the treatment film on the teeth. This helps the individual feel comfortable having the treatment film on their teeth for the prescribed amount of time to provide an effective treatment.

Moreover, when ethanol based compositions of poly(vinyl butyral-co-vinyl alcohol-co-vinyl acetate) are applied and dried on the teeth, the resulting treatment film maintains sufficient adhesiveness to the teeth to remain on the teeth for about two to about six hours. Thus, treatment varnishes according to the present invention allow for the active ingredient to leach into the teeth and/or saliva for a period of time that provides an effective treatment. Unlike conventional treatment varnishes that leave a hardened film that is difficult to remove from the teeth, the treatment varnish film of the present invention is easily removed in large layers simply by peeling the film off the teeth with a fingernail, toothbrush, or other appropriate tool. Thus the present invention saves time and hassle compared to conventional methods.

Furthermore, treatment varnishes derived from poly(vinyl butyral-co-vinyl alcohol-co-vinyl acetate) form a treatment film with an open polymer matrix. The open polymer matrix allows the active ingredients imbedded within the treatment film to dissolve and release from the treatment film in a time effective manner. Poly(vinyl butyral-co-vinyl alcohol-co-vinyl acetate) polymers are also non-toxic and biocompatible.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A treatment varnish composition for teeth, comprising:
ethanol;
one or more polymers that are soluble in ethanol, but are not soluble in water; and
one or more active ingredients, wherein:
the one or more polymers form a treatment film on the surface of teeth when the ethanol evaporates; and
the one or more active ingredients release from the treatment film during a period of time that the treatment film remains on the surface of the teeth.

2. The treatment varnish composition of claim 1, wherein the one or more polymers comprise poly vinyl butyral.

3. The treatment varnish composition of claim 1, wherein the one or more polymers comprise poly(vinyl butyral-co-vinyl alcohol-co-vinyl acetate).

4. The treatment varnish composition of claim 1, wherein the one or more polymers comprise at least one polymer selected from the group consisting of poly vinyl butyral, poly vinyl alcohol, or poly vinyl acetate.

5. The treatment varnish composition of claim 1, wherein the one or more polymers have a molecular weight greater than 150,000.

6. The treatment varnish composition of claim 1, wherein the one or more active ingredients includes one or more active ingredients selected from the group consisting of a fluoride, a remineralizer, a desensitizer, or a periodontal treatment.

7. The treatment varnish composition of claim 1, wherein the period of time in which the one or more active ingredients release from the treatment film is about two hours to about six hours.

8. The treatment varnish composition of claim 7, wherein the treatment film is easily removed from the teeth after the period of time.

9. The treatment varnish composition of claim 1, wherein the treatment film is a clear semi-translucent film.

10. The treatment varnish composition of claim 1, wherein the treatment film forms an open polymer matrix.

11. A polymer based composition made to coat teeth, comprising:
a biocompatible solvent;
a polymer that is soluble in the biocompatible solvent, but does not readily dissolve in water; and
one or more active ingredients that provide a treatment to teeth.

12. The polymer based composition of claim 11, wherein the biocompatible solvent is ethanol.

13. The polymer based composition of claim 12, wherein the polymer comprises poly(vinyl butyral-co-vinyl alcohol-co-vinyl acetate).

14. The polymer based composition of claim 13, wherein when the polymer based composition is applied to teeth, and the ethanol is evaporated, a clear semi-translucent treatment film is formed on the teeth.

15. The polymer based composition of claim 14, wherein the one or more active ingredients are released from the clear semi-translucent treatment film when exposed to saliva.

16. The polymer based composition of claim 15, wherein the clear semi-translucent treatment film is easily scrapped from the teeth after a treatment time of about two hours to about six hours.

17. A dental treatment composition, comprising:
a first state in which a polymer is dissolved in ethanol,
wherein the dental treatment composition in the first state can be applied to the surface of teeth;
a second state in which the ethanol is evaporated from the dental treatment composition,
wherein a treatment film that adheres to the teeth is formed upon the ethanol evaporating;
a third state in which the treatment film interacts with saliva,
wherein one or more active ingredients release from the treatment film upon interaction with saliva; and
a fourth state in which the treatment film's adhesion to the teeth declines after a treatment period of about two hours to about six hours,
wherein the treatment film can easily be removed from the teeth after the treatment period.

18. The dental treatment composition of claim 17, wherein the polymer that is dissolved in ethanol is poly(vinyl butyral-co-vinyl alcohol-co-vinyl acetate).

19. The dental treatment composition of claim 18, wherein the treatment film is a clear semi-translucent film.

20. The dental treatment composition of claim 19, wherein the one or more active ingredients includes one or more active ingredients selected from the group consisting of a fluoride, a remineralizer, a desensitizer, or a periodontal treatment.
